(19) United States
(12) Patent Application Publication
(10) Pub. No.: US 2013/0317034 A1
(43) Pub. Date: Nov. 28, 2013

(54) COMBINATION THERAPY FOR TREATING DIABETES

(71) Applicant: Japan Tobacco Inc., (US)

(72) Inventors: Yasuko Mera, Takatsuki (JP); Yukiharu Kuroki, Takatsuki (JP)

(73) Assignee: Japan Tobacco Inc., Tokyo (JP)

(21) Appl. No.: 13/862,071

(22) Filed: Apr. 12, 2013

Related U.S. Application Data
(63) Continuation of application No. PCT/IB2011/002432, filed on Oct. 14, 2011.
(60) Provisional application No. 61/393,042, filed on Oct. 14, 2010.

(51) Int. Cl. A61K 31/4985; A61K 31/216

(52) U.S. Cl. CPC A61K 31/4985; A61K 31/216
USPC 514/249; 514/533

(57) ABSTRACT

Provided is a method of treating or preventing diabetes in a subject comprising administering a therapeutically effective amount of (a) a compound of formula (I)

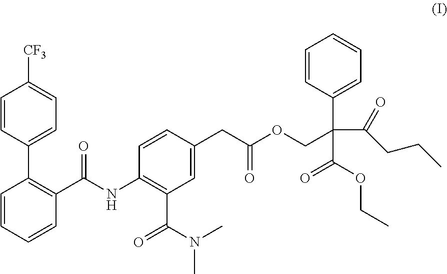 [SMILES: CCCC(=O)C(COC(=O)Cc1ccc(NC(=O)c2ccccc2-c2ccc(C(F)(F)F)cc2)c(C(=O)N(C)C)c1)(C(=O)OCC)c1ccccc1]

or a pharmaceutically acceptable salt thereof, and (b) a DPP-IV inhibitor, to a subject that has or is at risk of having diabetes, whereupon diabetes is treated or prevented in the subject. The method optionally comprises administering a therapeutically effective amount of the compound of formula (I), or a pharmaceutically acceptable salt thereof, to the subject, in which the therapeutically effective amount is increased once or twice during the course of administration.

### CROSS-REFERENCE TO RELATED APPLICATIONS

This patent application is a continuation of copending International Patent Application No. PCT/IB2011/002432, filed Oct. 14, 2011, which claims the benefit of U.S. Provisional Patent Application No. 61/393,042, filed Oct. 14, 2011, which are incorporated by reference in their entireties herein.

### BACKGROUND OF THE INVENTION

Diabetes refers to a disease process derived from multiple causative factors and characterized by elevated levels of plasma glucose or hyperglycemia in the fasting state or after administration of glucose during an oral glucose tolerance test. Persistent or uncontrolled hyperglycemia is associated with increased and premature morbidity and mortality.

Microsomal triglyceride transfer protein (MTP) exists in the microsome fractions of hepatocytes and intestinal epithelial cells, and regulates synthesis of triglyceride (TG) rich lipoproteins by assembly of TG and cholesteryl ester. MTP inhibition is believed to provide a treatment or prophylaxis of metabolic disorders, including hyperlipidemia, diabetes, and prediabetes.

Compounds that inhibit dipeptidyl peptidase-IV (“DP-IV” or “DPP-IV”) enzyme are drugs that are considered useful in the treatment of diabetes, particularly type 2 diabetes, because DPP-IV in vivo readily inactivates glucagon like peptide-1 (GLP-1). Inhibition of DPP-IV suppresses inactivation of GLP-1, which enhances glucose stimulated insulin secretion (GSIS).

Since some anti-diabetes agents are known to have side effects (e.g., increased hypoglycemia, weight gain, or hepatotoxicity), there is a need for methods of treating and/or preventing diabetes that are efficacious with a reduction or removal of the side effects associated with known treatments.

### BRIEF SUMMARY OF THE INVENTION

The invention provides a method of treating or preventing diabetes in a subject comprising, consisting essentially of, or consisting of administering a therapeutically effective amount of (a) a compound of formula (I)

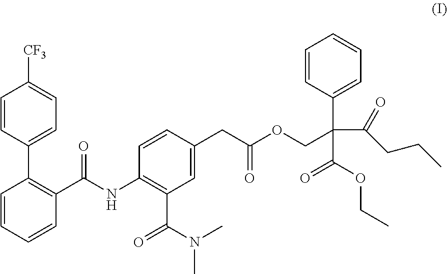 [SMILES: CCCC(=O)C(COC(=O)Cc1ccc(NC(=O)c2ccccc2-c2ccc(C(F)(F)F)cc2)c(C(=O)N(C)C)c1)(C(=O)OCC)c1ccccc1]

or a pharmaceutically acceptable salt thereof and (b) a dipeptidyl peptidase-IV (DPP-IV) inhibitor or a pharmaceutically acceptable salt thereof, to a subject that has or is at risk of having diabetes, whereupon diabetes is treated or prevented in the subject.

The method optionally comprises, consists essentially of, or consists of administering twice per day a therapeutically effective amount of the compound of formula (I), or a pharmaceutically acceptable salt thereof, to the subject. In one embodiment, the therapeutically effective amount is increased once or twice during the course of the administration of the compound of formula (I), or a pharmaceutically acceptable salt thereof. In another embodiment, the method comprises (a) administering twice per day a first therapeutically effective amount of the compound of formula (I), or a pharmaceutically acceptable salt thereof, to the subject for a first time period, and (b) administering twice per day a second therapeutically effective amount of the compound of formula (I), or a pharmaceutically acceptable salt thereof, to the subject for a second time period after the first time period, wherein the second therapeutically effective amount of the compound of formula (I) is greater than the first therapeutically effective amount of the compound of formula (I). In another embodiment, the method comprises (a) administering twice per day a first therapeutically effective amount of the compound of formula (I), or a pharmaceutically acceptable salt thereof, to the subject for a first time period, (b) administering twice per day a second therapeutically effective amount of the compound of formula (I), or a pharmaceutically acceptable salt thereof, to the subject for a second time period after the first time period, and (c) administering twice per day a third therapeutically effective amount of the compound of formula (I), or a pharmaceutically acceptable salt thereof, to the subject for a third time period after the second time period, wherein the second therapeutically effective amount of the compound of formula (I) is greater than the first therapeutically effective amount of the compound of formula (I), and the third therapeutically effective amount of the compound of formula (I) is greater than the second therapeutically effective amount of the compound of formula (I).

In certain embodiments, the invention provides that the compound is administered to the subject with food or when the subject is in a fed state.

### BRIEF DESCRIPTION OF THE SEVERAL VIEWS OF THE DRAWINGS

FIG. 1 is a bar graph illustrating the effect on plasma glucagon like peptide-1 (GLP-1) levels at the start of the glucose tolerance test (GTT). The white bar represents the control, the gray bar represents the administration of the compound of formula (I) only, the black and white striped bar represents the administration of sitagliptin only, and the black bar represents the combined administration of the compound of formula (I) and sitagliptin. Steel-Dwass test—**: p<0.01 versus control; ††: P<0.01 versus compound of formula (I); and ##: p<0.01 versus sitagliptin.

FIG. 2 is a graph illustrating the effect on plasma glucose levels in the GTT. ∘ represents the control, □ represents administration of the compound of formula (I) only, ▴ represents the administration of sitagliptin only, and ♦ represents the combined administration of the compound of formula (I) and sitagliptin. Tukey test—*: p<0.05; **: p<0.01 versus control; and ##: p<0.01 versus sitagliptin.

FIG. 3 is a graph illustrating the effect on plasma insulin levels in the GTT. ∘ represents the control, □ represents administration of the compound of formula (I) only, ▴ represents the administration of sitagliptin only, and ♦ represents the combined administration of the compound of formula (I) and sitagliptin. Tukey test—†: p<0.05 versus compound of formula (I).

### DETAILED DESCRIPTION OF THE INVENTION

The invention provides a method of treating or preventing diabetes in a subject comprising, consisting essentially of, or consisting of administering a therapeutically effective amount of (a) a compound of formula (I) or a pharmaceutically acceptable salt thereof and (b) a dipeptidyl peptidase-IV (DPP-IV) inhibitor or a pharmaceutically acceptable salt thereof, to a subject that has or is at risk of having diabetes, whereupon diabetes is treated or prevented in the subject.

The compound of formula (I), or a pharmaceutically acceptable salt thereof, is an MTP inhibitor.

The compound of formula (I) is chemically known as diethyl 2-((2-(3-(dimethylcarbamoyl)-4-(4′-(trifluoromethyl)biphenyl-2-ylcarboxamido)phenyl)acetoxy)methyl)-2-phenyl malonate. The compound of formula (I) can be prepared by any suitable synthesis, including the synthesis described in U.S. Patent Application Publication 2005/0075367, the entirety of which is incorporated herein by reference. As disclosed in Burnett (IDrugs, 9(7): 495-499 (2006)), the compound of formula (I) may be prepared, for example, according to the following procedure. 5-Chloro-2-nitrobenzoic acid is converted to its dimethylamide derivative via the acid chloride. The amide is treated with t-butyl methyl malonate to provide a 2-(4-nitrophenyl) malonate derivative. Selective hydrolysis and decarboxylation of the derivative yields the nitrophenyl acetic acid methyl ester, which can be reduced to its corresponding aniline. A reaction of the aniline with the acid chloride of 4′-trifluoromethyl-2-biphenylcarboxylic acid, and a hydrolysis of the ester, provide the biphenyl-2-carbonylaminophenyl acetic acid. Coupling of the acid with diethyl 2-(hydroxymethyl)-2-phenyl malonate in the presence of 4-dimethylaminopyridine and 1-ethyl-3-(3′-diethylaminopropyl)carbodiimide in dichloromethane at room temperature for 6 h produces the compound of formula (I). Additional information regarding the compound of formula (I) is set forth in International Patent Application PCT/US2010/038965, the entirety of which is incorporated herein by reference.

The DPP-IV inhibitor or a pharmaceutically acceptable salt thereof is any compound that is determined to block DPP-IV. Preferably, the DPP-IV inhibitor or a pharmaceutically acceptable salt thereof is a compound that does not cause undesirable side effects often associated with other anti-diabetic agents, such as hypoglycemia. Suitable examples of a DPP-IV inhibitor or a pharmaceutically acceptable salt thereof for use in the inventive methods include, e.g., sitagliptin (JANUVIA™), saxagliptin (ONGLYZA™) vildagliptin, linagliptin, dutogliptin, gemigliptin, teneligliptin, melogliptin, alogliptin, and compounds disclosed in U.S. Pat. Nos. 6,699,871 and 5,939,560, and International Patent Application Publications WO 1997/040832 and WO 1998/019998. Preferably, the DPP-IV inhibitor or a pharmaceutically acceptable salt thereof is a gliptin compound (e.g., sitagliptin, saxagliptin, vildagliptin, linagliptin, dutogliptin, gemigliptin, or alogliptin). In certain embodiments, the DPP-IV inhibitor is sitagliptin or a pharmaceutically acceptable salt thereof.

A “pharmaceutically acceptable salt” may be any kind of salt as long as it forms a nontoxic salt with the compound of formula (I) and/or the DPP-IV inhibitor and can be obtained by reacting the desired compound with, for example, an inorganic acid such as hydrochloric acid, sulfuric acid, phosphoric acid, or hydrobromic acid; an organic acid such as oxalic acid, malonic acid, citric acid, fumaric acid, lactic acid, malic acid, succinic acid, tartaric acid, acetic acid, trifluoroacetic acid, gluconic acid, ascorbic acid, methylsulfonic acid, or benzylsulfonic acid; an inorganic base such as sodium hydroxide, potassium hydroxide, calcium hydroxide, magnesium hydroxide, or ammonium hydroxide; an organic base such as methylamine, diethylamine, triethylamine, triethanolamine, ethylenediamine, tris(hydroxymethyl)methylamine, guanidine, choline, or cinchonine; or an amino acid such as lysine, arginine, or alanine.

The invention also includes solvent addition forms (“solvates”) of the compound of formula (I) or a pharmaceutically acceptable salt thereof, and/or the DPP-IV inhibitor or a pharmaceutically acceptable salt thereof. Some compounds have a tendency to trap a fixed molar ratio of solvent molecules in the crystalline solid state, thus forming a solvate. When the compound of formula (I) or a pharmaceutically acceptable salt thereof, and/or the DPP-IV inhibitor or a pharmaceutically acceptable salt thereof, is placed in a system in which a certain solvent is brought to a vapor form, in some situations, the compound, together with the molecules of the solvent, forms a crystal. The material formed by crystallization of the compound and the solvent in a three-dimensional order is called a solvate herein. The solvent can be associated with a crystalline solid form of the compound of formula (I) or a pharmaceutically acceptable salt thereof, and/or the DPP-IV inhibitor or a pharmaceutically acceptable salt thereof, in various ways. The interaction can be due to weak binding (e.g., hydrogen bonding, van der Waals, and dipole-dipole) or by entrapment (e.g., liquid inclusion).

A solvate can be formed by a variety of methods, many of which are known in the art. A compound of formula (I) or a pharmaceutically acceptable salt thereof, and/or the DPP-IV inhibitor or a pharmaceutically acceptable salt thereof, can be combined with one or more solvents by any suitable method (e.g., crystallization, lyophilization, film coating, spray drying, suspension, wetting, grinding, vapor sorption, etc.). For example, the compound of formula (I) or a pharmaceutically acceptable salt thereof, and/or the DPP-IV inhibitor or a pharmaceutically acceptable salt thereof, can be combined with a particular solvent(s) and heated to boiling. The solution can then be slowly cooled to allow formation of the solvate crystals. Cooling can occur at room temperature or at a reduced temperature (e.g., an ice bath and/or refrigerated conditions). Controlling the temperature can be influential in the formation of solvates. Typically a lower temperature favors solvate formation. The formed solvate can be characterized by analytical methods such as thermogravimetric analysis (TGA), differential scanning calorimetry (DSC) alone or with infrared (IR) and/or mass spectrometry, x-ray powder diffraction, moisture sorption experiments, hot-stage polarized light microscopy, or a combination of these methods. Various techniques to prepare solvates are known in the art.

A solvate means a solvent addition form that contains either stoichiometric or non-stoichiometric amounts of solvent. A stoichiometric solvate implies a fixed, although not necessarily integral, ratio of solvent to compound (e.g., a solvent coordination number of 1, 2, 3, 4, 5, 6, etc.). A preferred solvent coordination number of a stoichiometric solvate is 1. A non-stoichiometric solvate can be an interstitial solid solution or an interstitial co-crystal. The solvent content of a solvate can be any suitable value, including a multiple of the molar compound ratio such that the solvent coordination number is a non-integral number (e.g., 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, etc.). The amount of solvent in the structure generally depends on the partial pressure of the solvent in the environment of the solid and the temperature.

The solvent can be any suitable solvent, i.e., the solvent is not particularly limited as long as a solvate of the compound of formula (I) or a pharmaceutically acceptable salt thereof, and/or the DPP-IV inhibitor or a pharmaceutically acceptable salt thereof, can be formed. Solvents usable for solvate formation include water, alcohols, ethers, esters, alkanes, benzene, dichloromethane, chloroform, acetone, acetonitrile, toluene, tetrahydrofuran, pyridine, dimethylformamide (DMF), dimethyl sulfoxide (DMSO), dioxane, and combinations thereof. In some embodiments, the solvate contains a mixture of solvents, such as a combination of two or more of the aforementioned solvents. Preferably the solvent should have relatively low toxicity and can be removed from the compound of formula (I) or a pharmaceutically acceptable salt thereof, and/or the DPP-IV inhibitor or a pharmaceutically acceptable salt thereof, to a level that is acceptable according to The International Committee on Harmonization (ICH) guidelines (“ICH Q3C Impurities: Guideline for Residual Solvents, International Conference on Harmonization of Technical Requirements for Registration of Pharmaceuticals for Human Use,” Geneva, Switzerland, July 1997). Preferred solvents include water, alcohols, ethers, esters, and alkanes. If the solvent is water, the solvate formed is a “hydrate,” whereas when the solvent is alcohol, the solvate formed is an “alcoholate.” Specific examples of preferred solvents usable for solvate formation include water, C₁₋₄ alcohol (e.g., methanol, ethanol, propanol, isopropanol, and n-butanol), C₁₋₄ ether (e.g., diethyl ether), an ester of a C₁₋₆ (preferably C₁₋₄) alkyl acetate (e.g., methyl acetate, ethyl acetate, propyl acetate, and butyl acetate), a C₅₋₇ alkane (e.g., pentane, hexane, and heptane), and combinations thereof. Mixed solvates include, for example, water/ethanol, water/methanol, water/acetone, water/hexane, and water/DMF.

The method of treating or preventing diabetes in a subject can comprise, consist essentially of, or consist of administering a therapeutically effective amount of (a) a compound of formula (I) or a pharmaceutically acceptable salt thereof and (b) a dipeptidyl peptidase-IV (DPP-IV) inhibitor or a pharmaceutically acceptable salt thereof, to a subject that has or is at risk of having diabetes, whereupon diabetes is treated or prevented in the subject.

In certain embodiments, the method of treating or preventing diabetes in a subject comprises administering a therapeutically effective amount of (a) a compound of formula (I) or a pharmaceutically acceptable salt thereof and (b) a dipeptidyl peptidase-IV (DPP-IV) inhibitor or a pharmaceutically acceptable salt thereof, to a subject that has or is at risk of having diabetes, whereupon diabetes is treated or prevented in the subject. In such embodiments, the method can include the administration of any other compounds to the subject, including compounds that materially affect the inventive method (e.g., compounds that exert a biological effect when treating or preventing diabetes).

In other embodiments, the method of treating or preventing diabetes in a subject consists essentially of administering a therapeutically effective amount of (a) a compound of formula (I) or a pharmaceutically acceptable salt thereof and (b) a DPP-IV inhibitor or a pharmaceutically acceptable salt thereof, to a subject that has or is at risk of having diabetes, whereupon diabetes is treated or prevented in the subject. In such embodiments, the method precludes the administration of any compound other than (a) a compound of formula (I) or a pharmaceutically acceptable salt thereof and (b) a DPP-IV inhibitor or a pharmaceutically acceptable salt thereof, that materially affects the inventive method (e.g., compounds that exert a biological effect when treating or preventing diabetes). Thus, compounds that would be excluded in a method consisting essentially of administering (a) a compound of formula (I) or a pharmaceutically acceptable salt thereof and (b) a DPP-IV inhibitor or a pharmaceutically acceptable salt thereof are biologically active compounds, such as an anti-obesity agent, an anti-diabetic agent, an anti-dyslipdemic agent, an HMG CoA reductase inhibitor, an HMG CoA synthase inhibitor, a cholesterol absorption inhibitor (CAI), an acyl coenzyme A-cholesterol acyl transferase (ACAT) inhibitor, a CETP inhibitor, a squalene synthetase inhibitor, an anti-oxidant, a lipoprotein synthesis inhibitor, a renin angiotensin system inhibitor, a PPAR agonist, a PPAR antagonist, a triglyceride synthesis inhibitor, an anti-hypertensive agent, a fibrate, a CB1 antagonist, metformin, a PDE10A inhibitor, an insulin secretagogue, an α-glucosidase inhibitor, an insulin sensitizer, a hepatic glucose output lowering compound, insulin, an insulin derivative, and an aldosterone receptor agonist. Compounds that would not be excluded in a method consisting essentially of administering (a) a compound of formula (I) or a pharmaceutically acceptable salt thereof and (b) a DPP-IV inhibitor or a pharmaceutically acceptable salt thereof are compounds that do not materially affect the inventive method, such as carriers, excipients, vehicles, and other pharmaceutical additives (e.g., binders, lubricants, preservatives, colorings, and flavorings), as described herein, as well as biologically active compounds that do not exert a biological effect when treating or preventing diabetes.

In other embodiments, the method of treating or preventing diabetes in a subject consists essentially of administering a therapeutically effective amount of (a) a compound of formula (I) or a pharmaceutically acceptable salt thereof, (b) a DPP-IV inhibitor or a pharmaceutically acceptable salt thereof, and (c) one or more particular biologically active compound(s), such as metformin and other compounds disclosed herein, to a subject that has or is at risk of having diabetes, whereupon diabetes is treated or prevented in the subject. In such embodiments, the method precludes the administration of any compound other than (a) a compound of formula (I) or a pharmaceutically acceptable salt thereof, (b) a DPP-IV inhibitor or a pharmaceutically acceptable salt thereof, and (c) the one or more particular biologically active compound(s), that materially affects the inventive method (e.g., compounds that exert a biological effect when treating or preventing diabetes). Thus, compounds that would be excluded in a method consisting essentially of administering (a) a compound of formula (I) or a pharmaceutically acceptable salt thereof, (b) a DPP-IV inhibitor or a pharmaceutically acceptable salt thereof, and (c) the one or more particular biologically active compound(s), are biologically active compounds, such as an anti-obesity agent, an anti-diabetic agent, an anti-dyslipdemic agent, an HMG CoA reductase inhibitor, an HMG CoA synthase inhibitor, a cholesterol absorption inhibitor (CAI), an acyl coenzyme A-cholesterol acyl transferase (ACAT) inhibitor, a CETP inhibitor, a squalene synthetase inhibitor, an anti-oxidant, a lipoprotein synthesis inhibitor, a renin angiotensin system inhibitor, a PPAR agonist, a PPAR antagonist, a triglyceride synthesis inhibitor, an anti-hypertensive agent, a fibrate, a CB 1 antagonist, metformin, a PDE10A inhibitor, an insulin secretagogue, an α-glucosidase inhibitor, an insulin sensitizer, a hepatic glucose output lowering compound, insulin, an insulin derivative, and an aldosterone receptor agonist, to the extent such compounds are not the one or more particular biologically active compound(s). Compounds that would not be excluded in a method consisting essentially of administering a compound of formula (I) or a pharmaceutically acceptable salt thereof and a DPP-IV inhibitor or a pharmaceutically acceptable salt thereof are compounds that do not materially affect the inventive method, such as carriers, excipients, vehicles, and other pharmaceutical additives (e.g., binders, lubricants, preservatives, colorings, and flavorings), as described herein, as well as biologically active compounds that do not exert a biological effect when treating or preventing diabetes.

In other embodiments, the method of treating or preventing diabetes in a subject consists of administering a therapeutically effective amount of (a) a compound of formula (I) or a pharmaceutically acceptable salt thereof, (b) a DPP-IV inhibitor or a pharmaceutically acceptable salt thereof, optionally (c) one or more particular biologically active compound(s), such as metformin and other compounds disclosed herein, and optionally (d) one or more compounds that are not biologically active compounds, such as carriers, excipients, vehicles, and other pharmaceutical additives (e.g., binders, lubricants, preservatives, colorings, and flavorings), as described herein.

The compound of formula (I) or a pharmaceutically acceptable salt thereof, the DPP-IV inhibitor or a pharmaceutically acceptable salt thereof, and any other specified biologically active compound(s) are administered in one or more pharmaceutical compositions comprising, consisting essentially of, or consisting of (a) at least one active agent (i.e., the compound of formula (I) or a pharmaceutically acceptable salt thereof and/or the DPP-IV inhibitor or a pharmaceutically acceptable salt thereof and/or any other specified biologically active compound(s)) and (b) a pharmaceutically acceptable carrier.

Thus, the invention provides a pharmaceutical composition comprising, consisting essentially of, or consisting of (a) the compound of formula (I) or a pharmaceutically acceptable salt thereof, a DPP-IV inhibitor or a pharmaceutically acceptable salt thereof, and/or optionally any other specified biologically active compound(s) and (b) a pharmaceutically acceptable carrier. Typically, the pharmaceutical composition will comprise, consist essentially of, or consist of (a) the compound of formula (I) or a pharmaceutically acceptable salt thereof and/or a DPP-IV inhibitor or a pharmaceutically acceptable salt thereof and (b) a pharmaceutically acceptable carrier.

When the pharmaceutical composition consists essentially of (a) the compound of formula (I) or a pharmaceutically acceptable salt thereof and/or a DPP-IV inhibitor or a pharmaceutically acceptable salt thereof, and (b) a pharmaceutically acceptable carrier, compounds that exert a biological effect in a subject, such as those biologically active compounds described herein, are excluded from the composition. When the pharmaceutical composition consists of (a) the compound of formula (I) or a pharmaceutically acceptable salt thereof and/or a DPP-IV inhibitor or a pharmaceutically acceptable salt thereof, and (b) a pharmaceutically acceptable carrier, the composition excludes any other compounds.

Pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

If desired, pharmaceutical additives such as an excipient, a lubricant, a binder, a disintegrating agent, a solvent, a solubilizer, a suspending agent, an isotonizing agent, a buffer, a soothing agent, a preservative, an antioxidant, a sweetening agent, or a coloring agent can also be used. Suitable examples of an excipient include lactose, sucrose, D-mannitol, starch, crystalline cellulose, and light anhydrous silicic acid. Suitable examples of a lubricant include magnesium stearate, calcium stearate, talc, and colloidal silica. Suitable examples of a binder include crystalline cellulose, sucrose, D-mannitol, dextrin, hydroxypropyl cellulose, hydroxypropyl methylcellulose, and polyvinylpyrrolidone. Suitable examples of a disintegrating agent include starch, carboxymethylcellulose, carboxymethylcellulose calcium, crosscarmellose sodium, and sodium carboxymethylstarch. Suitable examples of a solvent include purified water, alcohol, propylene glycol, macrogol, sesame-seed oil, corn oil, and propylene glycol fatty acid ester. Suitable examples of a solubilizer include polyethyleneglycol, propyleneglycol, D-mannitol, benzyl benzoate, ethanol, trisaminomethane, cholesterol, triethanolamine, sodium carbonate, and sodium citrate. Suitable examples of a suspending agent include surfactants (e.g., stearyl triethanolamine, sodium lauryl sulfate, lauryl aminopropionic acid, lecithin, benzalkonium chloride, benzethonium chloride, and glycerin monostearate), polyvinyl alcohol, polyvinylpyrrolidone, carboxymethylcellulose sodium, methylcellulose, and hydroxymethyl cellulose. Suitable examples of an isotonizing agent include sodium chloride, glycerin, and D-mannitol. Suitable examples of a buffer include phosphate, acetate, carbonate, and citrate. Suitable examples of a soothing agent include benzyl alcohol. Suitable examples of a preservative include paraoxybenzoic acid esters, chlorobutanol, benzyl alcohol, phenethyl alcohol, dehydroacetic acid, and sorbic acid. Suitable examples of an antioxidant include sulfites and ascorbic acid. Suitable examples of a sweetening agent include aspartame, saccharin sodium, and stevia. Suitable examples of a coloring agent include food colors such as Food Yellow No. 5, Food Red No. 2, and Food Blue No. 2, lake colors for food, and iron oxide.

A pharmaceutical composition comprising, consisting essentially of, or consisting of (a) the compound of formula (I), or a pharmaceutically acceptable salt thereof and/or (b) the DPP-IV inhibitor or a pharmaceutically acceptable salt thereof can be provided in any suitable formulation, such as tablets, capsules, pills, powders, granules, solutions, capsules, troches, elixirs, suspensions, emulsions, and syrups. In certain embodiments, the pharmaceutical composition is administered as an oral formulation, such as a tablet or capsule. In a preferred embodiment, the oral formulation is a tablet (e.g., a tablet prepared in accordance with the methods and compositions described in U.S. Patent Application Publication 2006/0153913A1, the entire contents of which are incorporated herein by reference).

When the compound of formula (I) or a pharmaceutically acceptable salt thereof, and/or the DPP-IV inhibitor or a pharmaceutically acceptable salt thereof, is formulated into a solid preparation, such as a tablet, pill, powder, or granules, additives such as lactose, mannitol, glucose, hydroxypropyl cellulose, microcrystalline cellulose, starch, polyvinylpyrrolidone, magnesium aluminometasilicate, or powdery silicic anhydride can be used. When the compound of formula (I) or a pharmaceutically acceptable salt thereof, and/or the DPP-IV inhibitor or a pharmaceutically acceptable salt thereof, is formulated into a tablet or pill, it can be coated with a gastroenteric or enteric coating film containing a substance such as sucrose, gelatin, hydroxypropyl cellulose, or hydroxymethyl cellulose phthalate. Furthermore, the tablet or pill can be a multi-layered tablet or pill comprising two or more layers.

Suitable oral formulations include capsules that comprise liquid, semi-solid, or solid contents. Liquid or semi-solid contents can be prepared by dissolving the compound of formula (I) or a pharmaceutically acceptable salt thereof, and/or the DPP-IV inhibitor or a pharmaceutically acceptable salt thereof, in a solvent and adding an additive thereto. Examples of solvents are purified water, ethanol, and vegetable oil. Ethanol or a mixture of purified water and ethanol preferably is used. Suitable additives for a capsule include, for example, propylene glycol fatty acid esters; low molecular weight polyethylene glycols such as polyethylene glycol 200 to 600 g/mol, glycerine fatty acid esters thereof, and medium chain fatty acid triglycerides thereof; alcohols/polyols such as stearyl alcohol, cetanol, polyethylene glycol, and esters thereof; lipids such as sesame oil, soy bean oil, peanut oil, corn oil, hydrogenated oil, paraffin oil, and bleached wax; and fatty acids such as triethyl citrate, triacetin, stearic acid, palmitic acid, and myristic acid. These additives are suitable for preparing liquid or semi-solid contents of the capsule.

Propylene glycol fatty acid esters are a preferable additive in capsule formulations. Examples of the propylene glycol fatty acid esters are propylene glycol monocaprylate (CAPMUL™ PG-8, SEFOL™ 218, and CAPRYO™ 190), propylene glycol monolaurate (LAUROGLYCOL™ FCC), propylene glycol monooleate (MYVEROL™ P-06), propylene glycol myristate, propylene glycol monostearate, propylene glycol lisinolate (PROPYMULS™), propylene glycol dicaprylate/dicaprate (CAPTEX™ 200) propylene glycol dilaurate, propylene glycol distearate, and propylene glycol dioctanoate (CAPTEX™ 800). Although there is no particular limitation to the materials constituting the capsules of the invention, the materials can include, for example, polysaccharides derived from natural products such as agar, alginic acid salt, starch, xanthan, and dextran; proteins such as gelatin and casein; chemically processed products such as hydroxystarch, pullulan, hydroxypropyl cellulose, hydroxypropyl methylcellulose, polyvinyl alcohol and derivatives thereof, polyacryl derivatives, polyvinylpyrrolidone and derivatives thereof, and polyethylene glycol.

When the pharmaceutical composition is a liquid formulation for oral administration, such as a pharmaceutically acceptable emulsion, solubilizer, suspension, syrup, or elixir, suitable diluents include, for example, purified water, ethanol, vegetable oils, and emulsifiers. In addition, auxiliary agents such as wetting agents, suspending agents, sweeteners, condiments, flavors, and antiseptics can be added to a liquid formulation.

There is no particular limitation as to the timing for the administration of the compound of formula (I) or a pharmaceutically acceptable salt thereof, and the DPP-IV inhibitor or a pharmaceutically acceptable salt thereof. For example, the DPP-IV inhibitor or a pharmaceutically acceptable salt thereof can be administered simultaneously, substantially simultaneously (e.g., within 1 hour, within 45 min, within 30 min, within 20 min, within 15 min, within 10 min, or within 5 min of each other), or intermittently (e.g., before and/or after) with the compound of formula (I) or a pharmaceutically acceptable salt thereof.

The phrase “therapeutically effective amount” means an amount of the compound of formula (I) or a pharmaceutically acceptable salt thereof, or the DPP-IV inhibitor or a pharmaceutically acceptable salt thereof, that (i) treats or prevents diabetes, (ii) attenuates, ameliorates, or eliminates one or more symptoms of diabetes, and/or (iii) prevents or delays the onset of one or more symptoms of diabetes. In the context of numerical values for dosages as provided herein, the term “about” means±0.25 mg of any recited amount or range.

The dosage of the compound of formula (I) or a pharmaceutically acceptable salt thereof can be determined based on clinical doses, and can be chosen depending on the age, weight, and/or condition of the subject, medication time, dosage form, method of administration, or any combination of the foregoing factors.

In particular, the therapeutically effective amount of the compound of formula (I), or a pharmaceutically acceptable salt thereof, is about 1 mg or more (e.g., about 1.5 mg or more, about 2 mg or more, about 2.5 mg or more, about 3 mg or more, about 3.5 mg or more, about 4 mg or more, about 4.5 mg or more, about 5 mg or more, about 5.5 mg or more, about 6 mg or more, about 6.5 mg or more, about 7 mg or more, about 7.5 mg or more, about 8 mg or more, about 8.5 mg or more, about 9 mg or more, about 9.5 mg or more, or about 10 mg or more) per day. Alternatively, or in addition, the therapeutically effective amount of the compound of formula (I), or a pharmaceutically acceptable salt thereof, is about 20 mg or less (e.g., about 19.5 mg or less, about 19 mg or less, about 18.5 mg or less, about 18 mg or less, about 17.5 mg or less, about 17 mg or less, about 16.5 mg or less, about 16 mg or less, about 15.5 mg or less, about 15 mg or less, about 14.5 mg or less, about 14 mg or less, about 13.5 mg or less, about 13 mg or less, about 12.5 mg or less, about 12 mg or less, about 11.5 mg or less, about 11 mg or less, about 10.5 mg or less, about 10 mg or less, about 9.5 mg or less, about 9 mg or less, about 8.5 mg or less, about 8 mg or less, about 7.5 mg or less, about 7 mg or less, about 6.5 mg or less, about 6 mg or less, about 5.5 mg or less, or about 5 mg or less) per day. Thus, the therapeutically effective amount of the compound of formula (I), or a pharmaceutically acceptable salt thereof, can be a range bounded by any two of the aforementioned endpoints.

For example, the therapeutically effective amount of the compound of formula (I), or a pharmaceutically acceptable salt thereof, can be about 1 mg to about 20 mg (e.g., about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, or about 20 mg) per day. The daily dose of the compound of formula (I) or a pharmaceutically acceptable salt thereof can be administered to a subject once per day or alternatively as two, three, four, or more sub-doses administered separately at appropriate intervals throughout the day, optionally, in unit dosage forms. Each such sub-dose preferably contains a therapeutically effective amount of the compound of formula (I) or a pharmaceutically acceptable salt thereof. The combination, pharmaceutical composition, package, and kit, as described herein, can contain any suitable amount of the compound of formula (I) or a pharmaceutically acceptable salt thereof, and typically will contain the aforementioned total amount or sub-dose to be administered to a subject per day.

The compound of formula (I) or a pharmaceutically acceptable salt thereof desirably is administered to a subject once or twice per day, preferably twice per day (typically in equal therapeutically effective amounts). Preferably, the compound of formula (I), or a pharmaceutically acceptable salt thereof, is administered to a subject in an amount of about 0.5 mg to about 10 mg (e.g., about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, or about 10 mg) twice per day. The total amount of the compound of formula (I) or a pharmaceutically acceptable salt thereof to be administered to a subject per day generally should not exceed 20 mg (e.g., no more than two equal doses of 10 mg each day).

The dosage of the DPP-IV inhibitor or a pharmaceutically acceptable salt thereof can be determined based on clinical doses, and can be chosen depending on the age, weight, and/or condition of the subject, medication time, dosage form, method of administration, or any combination of the foregoing factors. In certain embodiments, the therapeutically effective amount of the DPP-IV inhibitor or a pharmaceutically acceptable salt thereof is about 1 mg to about 150 mg (e.g., about 1, 2, 2.5, 5, 10, 25, 50, 75, 100, 125, or 150 mg) per dose, which is administered any suitable number of times per day, preferably once or twice per day. In a preferred embodiment of the invention, the DPP-IV inhibitor or a pharmaceutically acceptable salt thereof is administered once per day. In certain embodiments, the therapeutically effective amount of the DPP-IV inhibitor or a pharmaceutically acceptable salt thereof is about 2.5 mg to about 100 mg (e.g., about 2.5, 5, 10, 25, 50, 75, or 100 mg) per day. Preferably, the therapeutically effective amount of the DPP-IV inhibitor or a pharmaceutically acceptable salt thereof is about 25 mg, about 50 mg, or about 100 mg, which is administered once per day. In accordance with the inventive method, the DPP-IV inhibitor or a pharmaceutically acceptable salt thereof can be administered with or without food.

If desired, the daily dose of the DPP-IV inhibitor or a pharmaceutically acceptable salt thereof can be administered to a subject once per day or alternatively as two, three, four, or more sub-doses administered separately at appropriate intervals throughout the day, optionally, in unit dosage forms. Each such sub-dose preferably contains a therapeutically effective amount of the DPP-IV inhibitor or a pharmaceutically acceptable salt thereof. The combination, pharmaceutical composition, package, and kit, as described herein, can contain any suitable amount of the DPP-IV inhibitor or a pharmaceutically acceptable salt thereof, and typically will contain the aforementioned total amount or sub-dose to be administered to a subject per day.

The compound of formula (I) or a pharmaceutically acceptable salt thereof can be administered to a subject at any suitable interval. The DPP-IV inhibitor or a pharmaceutically acceptable salt thereof also can be administered to a subject at any suitable interval, which may be the same or different than the interval for administration of the compound of formula (I) or a pharmaceutically acceptable salt thereof.

The inventive method can comprise, consist essentially of, or consist of administering once or twice per day a therapeutically effective amount of the compound of formula (I), or a pharmaceutically acceptable salt thereof, to the subject. Preferably, the method comprises, consists essentially of, or consists of administering twice per day a therapeutically effective amount of the compound of formula (I), or a pharmaceutically acceptable salt thereof, to the subject.

The inventive method can comprise, consist essentially of, or consist of administering once or twice per day a therapeutically effective amount of the DPP-IV inhibitor, or a pharmaceutically acceptable salt thereof, to the subject. Preferably, the method comprises, consists essentially of, or consists of administering once per day a therapeutically effective amount of the DPP-IV inhibitor, or a pharmaceutically acceptable salt thereof, to the subject.

In an embodiment of the invention, the method contemplates an escalating dosing schedule. Typically, the method comprises administering twice per day a therapeutically effective amount of the compound of formula (I), or a pharmaceutically acceptable salt thereof, to the subject, and the therapeutically effective amount is increased once or twice during the course of the administration of the compound of formula (I), or a pharmaceutically acceptable salt thereof. For example, the method comprises (a) administering a first therapeutically effective amount of the compound of formula (I), or a pharmaceutically acceptable salt thereof, to the subject for a first time period, and (b) administering a second therapeutically effective amount of the compound of formula (I), or a pharmaceutically acceptable salt thereof, to the subject for a second time period after the first time period. The method can optionally comprise step (c) administering a third therapeutically effective amount of the compound of formula (I), or a pharmaceutically acceptable salt thereof, to the subject for a third time period after the second time period. The second therapeutically effective amount is greater than the first therapeutically effective amount, and if used, the third therapeutically effective amount is greater than the second therapeutically effective amount. Typically, the first and second time periods are the same or different and each independently is about 1 to 5 weeks (i.e., about 1 week, about 2 weeks, about 3 weeks, about 4 weeks, or about 5 weeks). Preferably the first and/or second time period independently is about 2 to 4 weeks (e.g., about 2 weeks, about 3 weeks, or about 4 weeks). More preferably, the first and/or second time period is about 2 weeks. The term “about” in this context of time means±2 days of any given time or range of times.

Preferably, the first time period and the second time period abut one another, such that the beginning of the second time period immediately follows the end of the first time period. In certain embodiments, however, there is a time lag between the first and second time periods, such that there is an interval of time between the end of the first time period and the beginning of the second time period. This interval of time can vary depending on the subject and the prognosis of the treatment method, but typically the interval of time between the first and second time period is at least one day (e.g., at least two days, at least 3 days, at least 4 days, at least 5 days, at least 6 days, at least 7 days) but no more than 2 weeks (e.g., less than 10 days, less than 7 days, less than 6 days, less than 5 days, less than 4 days, less than 3 days).

In a method with an escalating dosing schedule, the first therapeutically effective amount of the compound of formula (I), or a pharmaceutically acceptable salt thereof, can be any therapeutically effective amount disclosed herein. The first therapeutically effective amount typically is about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg per day. The second therapeutically effective amount typically is at least 25% greater, at least 50% greater, at least 75% greater, or at least 100% greater than the first therapeutically effective amount. If a third dose is contemplated, the third therapeutically effective amount is at least 25% greater, at least 50% greater, at least 75% greater, or at least 100% greater than the second therapeutically effective amount. The total amount of the compound of formula (I) or a pharmaceutically acceptable salt to be administered to a subject per day, even after one or more dose escalations, generally should not exceed 20 mg (e.g., no more than two equal doses of 10 mg each day).

The compound of formula (I), or a pharmaceutically acceptable salt thereof, and the DPP-IV inhibitor, or a pharmaceutically acceptable salt thereof, desirably are administered to a subject in the form of one or more pharmaceutical compositions.

The pharmaceutical composition of the invention can be in the form of a package that contains separate dosage units, comprising, consisting essentially of, or consisting of (a) at least one dosage unit comprising the compound of formula (I) or a pharmaceutically acceptable salt thereof, and (b) at least one other dosage unit comprising a DPP-IV inhibitor or a pharmaceutically acceptable salt thereof. Each of the separate dosage units can comprise the aforementioned active ingredient of that separate dosage unit with one or more pharmaceutically acceptable carriers. A “package” is understood to be any package useful for stable storage of the dosage units. The package may, for example, be a glass or plastic (e.g., a high-density polyethylene) container generally used for packaging and storage of tablets or a blister pack.

A kit of the invention comprises (a) a first pharmaceutical composition comprising, consisting essentially of, or consisting of a therapeutically effective amount of (i) the compound of formula (I) or a pharmaceutically acceptable salt thereof and (ii) a pharmaceutically acceptable carrier, (b) a second pharmaceutical composition comprising, consisting essentially of, or consisting of (i) a DPP-IV inhibitor or a pharmaceutically acceptable salt thereof and (ii) a pharmaceutically acceptable carrier, (c) prescribing information, and (d) a container. Alternatively, a kit can comprise (a) a first pharmaceutical composition comprising, consisting essentially of, or consisting of a therapeutically effective amount of (i) the compound of formula (I) or a pharmaceutically acceptable salt thereof, (ii) a DPP-IV inhibitor or a pharmaceutically acceptable salt thereof, and (iii) a pharmaceutically acceptable carrier, (b) a second pharmaceutical composition comprising, consisting essentially of, or consisting of (i) the compound of formula (I) or a pharmaceutically acceptable salt thereof, and (ii) a pharmaceutically acceptable carrier, (c) prescribing information, and (d) a container. The prescribing information includes advice to a subject (e.g., patient) regarding co-administration of (a) the compound of formula (I) or a pharmaceutically acceptable salt thereof, and (b) the DPP-IV inhibitor or a pharmaceutically acceptable salt thereof. The prescribing information desirably advises the subject (e.g., patient) to administer within a 24-hour period (a) the compound of formula (I) or a pharmaceutically acceptable salt thereof twice to a subject, and (b) the DPP-IV inhibitor or a pharmaceutically acceptable salt thereof once to a subject.

The first and second pharmaceutical compositions can be the same (i.e., a single pharmaceutical composition) or different (i.e., two separate pharmaceutical compositions). For example, in some instances, the first and second pharmaceutical compositions are the same, i.e., are administered as a single dosage unit (e.g., tablet) and can comprise, consist essentially of, or consist of (i) the compound of formula (I) or a pharmaceutically acceptable salt thereof, (ii) a DPP-IV inhibitor or a pharmaceutically acceptable salt thereof, and (iii) a pharmaceutically acceptable carrier. In other instances, the first and second pharmaceutical compositions are different, i.e., are administered as separate dosage units (e.g., tablets). In such instances, the first pharmaceutical composition can comprise, consist essentially of, or consist of (i) the compound of formula (I) or a pharmaceutically acceptable salt thereof, (ii) a DPP-IV inhibitor or a pharmaceutically acceptable salt thereof, and (iii) a pharmaceutically acceptable carrier, whereas the second pharmaceutical composition can comprise, consist essentially of, or consist of (i) the compound of formula (I) or a pharmaceutically acceptable salt thereof and (ii) a pharmaceutically acceptable carrier.

Since the compound of formula (I), or a pharmaceutically acceptable salt thereof, preferably is administered twice per day to a subject, while the DPP-IV inhibitor, or a pharmaceutically acceptable salt thereof, preferably is administered once per day to the subject, two different pharmaceutical compositions are utilized and administered to the subject: (1) a first pharmaceutical composition comprising, consisting essentially of, or consisting of (i) the compound of formula (I) or a pharmaceutically acceptable salt thereof, (ii) a DPP-IV inhibitor or a pharmaceutically acceptable salt thereof, (iii) optionally one or more other biologically active compounds, and (iv) a pharmaceutically acceptable carrier, and (2) a second pharmaceutical composition comprising, consisting essentially of, or consisting of (i) the compound of formula (I) or a pharmaceutically acceptable salt thereof, (ii) optionally one or more other biologically active compounds, and (iii) a pharmaceutically acceptable carrier. In other words, preferably, the first pharmaceutical composition to be administered each day to a subject includes both the compound of formula (I), or a pharmaceutically acceptable salt thereof, and a DPP-IV inhibitor, or a pharmaceutically acceptable salt thereof, while the second pharmaceutical composition to be administered each day to the subject does includes the compound of formula (I), or a pharmaceutically acceptable salt thereof, but does not include a DPP-IV inhibitor, or a pharmaceutically acceptable salt thereof.

The container in the kit also can provide means for separating the first and second pharmaceutical compositions. For example, the container can be a divided bottle or a divided foil packet (e.g., blister pack). Typically the kit comprises directions for the administration of the separate components. Optionally, a dispenser designed to dispense the daily doses one at a time in the order of their intended use is provided. Preferably, the dispenser is equipped with a memory aid, so as to further facilitate compliance with the regimen. For example, the memory aid may be a mechanical counter that indicates the number of daily doses that has been dispensed. Another example of such a memory-aid is a battery-powered microchip memory coupled with a liquid crystal readout, or audible reminder signal which, for example, reads out the date that the last daily dose has been taken and/or reminds one when the next dose is to be taken.

In an embodiment of the inventive method, at least the compound of formula (I), or a pharmaceutically acceptable salt thereof, is administered to the subject with food or when the subject is in a fed state. The term “fed state” means the subject has commenced the consumption of food within about 180 minutes prior to administering the compound of formula (I) or a pharmaceutically acceptable salt thereof. In some instances, the compound is administered to the subject with food. In other instances, the compound is administered to the subject after the subject has begun to consume food (e.g., about 1-180 minutes after the subject has begun to eat). The term “about” in the context of time relative to the consumption of food means±5 minutes of any given time or range of times. The compound of formula (I), or a pharmaceutically acceptable salt thereof, can be administered to the subject about 5 minutes, about 10 minutes, about 20 minutes, or about 30 minutes after the subject has begun to consume food. Alternatively, or in addition, the compound of formula (I), or a pharmaceutically acceptable salt thereof, can be administered to the subject no more than about 180 minutes, no more than about 120 minutes, no more than about 60 minutes, or no more than about 30 minutes after the subject has begun to consume food. Thus, the compound of formula (I), or a pharmaceutically acceptable salt thereof, can be administered in any time period bounded by any two of the foregoing values, e.g., about 5-180 minutes, about 20-180 minutes, about 30-180 minutes, about 10-120 minutes, about 20-120 minutes, about 20-60 minutes, or about 30-60 minutes. In an especially preferred embodiment, the compound of formula (I), or a pharmaceutically acceptable salt thereof, is administered about 30 minutes after the subject has begun to consume food. Since the compound of formula (I), or a pharmaceutically acceptable salt thereof, is administered twice per day, in a preferred embodiment, the compound is administered either with or subsequent to consuming food in time periods that are no less than about four hours (e.g., more than about 4 h, more than about 5 h, more than about 6 h, more than about 7 h, more than about 8 h, more than about 9 h, more than about 10 h) apart. The food desirably is solid food, especially solid food of at least 30 g (e.g., at least 40 g, at least 50 g, at least 75 g, at least 100 g, at least 125 g, or at least 150 g). Preferably the food is part of a meal (e.g., breakfast, lunch, or dinner). For example, the compound of formula (I), or a pharmaceutically acceptable salt thereof, can be administered to the subject either with or after consuming breakfast and dinner, in which breakfast and dinner are at least about four hours apart.

The invention provides a therapeutic agent for the treatment or prophylaxis of diabetes. The term “diabetes” includes disorders commonly understood to correspond to diabetes, such as prediabetes, increased plasma glucose level (hyperglycemia), and insulin resistance. Preferably, the diabetes is type 2 diabetes. Preferably, one or more symptoms of diabetes is prevented, reduced, or eliminated subsequent to administration of the compound of formula (I), or a pharmaceutically acceptable salt thereof, and the DPP-IV inhibitor or a pharmaceutically acceptable salt thereof, thereby effectively treating or preventing diabetes to at least some degree. The term “treating” means at least an amelioration of the symptoms associated with diabetes. The term “amelioration” is used in a broad sense to refer to at least a reduction in the magnitude of a parameter, e.g., symptom, associated with diabetes, such as elevated plasma glucose and/or insulin levels. As such, “treating” also includes situations where diabetes, or at least symptoms associated therewith, are completely inhibited (e.g., prevented from happening) or stopped (e.g., terminated) such that the subject no longer suffers from diabetes, or at least the symptoms that characterize diabetes (e.g., plasma glucose and/or insulin levels are returned to normal). The treatment of diabetes (e.g., type 2 diabetes) can be measured as maintaining blood glucose levels within acceptable ranges for a given subject.

Prediabetes is characterized by impaired fasting glucose (IFG) and/or impaired glucose tolerance (IGT). Most individuals with IFG or IGT will develop progressive hyperglycemia, eventually meeting the criteria for type 2 diabetes. Individuals with prediabetes typically have a high cardiovascular disease (CVD) risk as well (e.g., reported hazard ratio 1.1-1.4). IFG is defined by an elevated fasting plasma glucose (FPG) concentration (≧100 and <126 mg/dL). IGT is defined by an elevated 2 hr plasma glucose concentration (≧140 and <200 mg/dL) after a 75 g glucose load on the oral glucose tolerance test (OGTT) in the presence of an FPG concentration <126 mg/dL. The treatment of prediabetes can be measured as maintaining blood glucose levels within acceptable ranges for a given subject.

The compound of formula (I) or a pharmaceutically acceptable salt thereof and the DPP-IV inhibitor or a pharmaceutically acceptable salt thereof, is administered to a subject, such as a mammal. Suitable mammals are from the order Carnivora, including Felines (cats) and Canines (dogs), the order Artiodactyla, including Bovines (cows) and Swines (pigs), the order Perssodactyla, including Equines (horses), the order Primates, Ceboids, or Simioids (monkeys), or the order Anthropoids (humans and apes). An especially preferred mammal is the human. In certain embodiments, the human is an obese human, a human with type 2 diabetes, an obese human with type 2 diabetes, a human with prediabetes, and/or an obese human with prediabetes.

In another embodiment, the compound of formula (I) is indicated as an adjunct to diet and exercise to improve glycemic control in a patient with type 2 diabetes when administered to a patient in need thereof according to one or more methods of the invention.

In another embodiment, the compound of formula (I) is indicated as an adjunct to diet, exercise, and/or a reduced calorie diet (i) to improve glycemic control, and (ii) for obesity management, including weight loss and weight maintenance, when administered to a patient in need thereof (e.g., an obese type 2 diabetes patient or an obese prediabetes patient) according to one or more methods of the invention.

In another embodiment of the invention, the compound of formula (I) is indicated to treat IFG and/or IGT when administered to a patient in need thereof (e.g., a prediabetes patient or an obese prediabetes patient) according to one or more methods of the invention.

In another embodiment of the invention, the compound of formula (I) is indicated to reduce the risk for diabetes when administered to a patient in need thereof (e.g., a prediabetes patient, an obese prediabetes patient, or an obese patient) according to one or more methods of the invention.

As used herein, the term “patient” is a subject, as defined herein. Preferably the patient is a human and can be an infant, child, adolescent, or adult. Preferably, the patient is an adult human.

### Example

This example further illustrates the invention but, of course, should not be construed as in any way limiting its scope.

This example studied the combined effects of the compound of formula (I) and sitagliptin, i.e., a DPP-IV inhibitor, on plasma GLP-1 levels and glucose tolerance as evaluated in rats fed a high fat diet.

Forty-four eight week old male rats (Charles River Laboratories Japan, Inc.) were used for the experiment. Body weights and food weight were measured six days before and on the day before the dosing of the compound of formula (I). Body weight gain and food consumption were calculated for this period. Animals were fasted for 24 hours the day before dosing with the compound of formula (I). Using the body weights on the day of the dosing, body weight gain and food consumption as indicators, 32 rats were selected from those closest to the mean values. The rats were assigned to four groups (control, compound of formula (I) only, DPP-IV inhibitor only, and combination of compound of formula (I) and DPP-IV inhibitor) with eight animals in each group. One-way analysis of variance was performed using SAS Preclinical Package Version 5.0 and SAS System Version 8.2 (SAS Institute Japan Co., Ltd.) to verify that no significant intergroup differences were present after group allocation.

To prepare the vehicle, methylcellulose (MC) powder (METOLOSE™ SM-1500, Shin-Etsu Chemical Co., Ltd.) was dissolved in purified water to prepare a 0.5% solution. The solution was used within four days of preparation. At the time of use, the compound of formula (I) was suspended in the vehicle dosing solution at a concentration of 15 mg/mL. Sitagliptin phosphate monohydrate (hereafter sitagliptin) was suspended in the vehicle dosing solution at a concentration of 5 mg/mL immediately prior to use.

After the rats were fasted for 24 hours, either the compound of formula (I) (30 mg/kg) or vehicle (0.5% MC) was dosed orally. The rats were immediately fed with a high-fat diet for six hours and fasted overnight. Glucose tolerance tests (GTTs) were performed 30 minutes after dosing either sitagliptin or vehicle (0.5% MC). Dosing for each group is described in Tables 1 and 2 below.

TABLE 1
Dosing of Test Article

| | | | | Concen- | | |
|---|---|---|---|---|---|---|
| | | Dose of | | tration | | |
| | Test | Test | | of Test | | |
| | Article or | Article | Volume | Article | Dosing | No. of |
| Group | Vehicle | (mg/kg) | (mL/kg) | (mg/mL) | Route | Animals |
| control | vehicle | 0 | 2.0 | 0 | PO | 8 |
| compound | compound | 30 | 2.0 | 15 | PO | 8 |
| of formula | of formula | | | | | |
| (I) | (I) | | | | | |
| sitagliptin | vehicle | 0 | 2.0 | 0 | PO | 8 |
| combina- | compound | 30 | 2.0 | 15 | PO | 8 |
| tion | of formula | | | | | |
| | (I) | | | | | |

TABLE 2
Dosing of the Concomitantly Administered Article

| | | | | Concentration | | |
|---|---|---|---|---|---|---|
| | | Dose of | | of | | |
| | Concomitant | Concomitant | | Concomitant | | |
| | Article or | Article | Volume | Article | Dosing | No. of |
| Group | Vehicle | (mg/kg) | (mL/kg) | (mg/mL) | Route | Animals |
| control | vehicle | 0 | 2.0 | 0 | PO | 8 |
| compound of | vehicle | 0 | 2.0 | 0 | PO | 8 |
| formula (I) | | | | | | |
| sitagliptin | sitagliptin | 10 | 2.0 | 5 | PO | 8 |
| combination | sitagliptin | 10 | 2.0 | 5 | PO | 8 |

Food consumption was calculated according to the following formula: food consumption (g)=food weight before feeding (g)−food weight after feeding (g). Body weight gain was calculated according the following formula: body weight gain (g)=body weight after feeding (g)-body weight before feeding (g). There were no significant differences between the groups in food consumption and body weight gain for six hours after dosing, and in body weight at the start of GTT (Table 3).

TABLE 3
Effects on Food Consumption and Body Weight Gain
Six House After Feeding - Body Weights Before GTT

| | Food | Body | |
|---|---|---|---|
| Group | Consumption (g) | Weight Gain (g) | Body Weight (g) |
| control | 13.7 ± 0.6 | 14.6 ± 1.2 | 350.7 ± 6.6 |
| compound of | 11.9 ± 0.7 | 10.9 ± 0.9 | 348.9 ± 7.1 |
| formula (I) | | | |
| sitagliptin | 15.2 ± 1.3 | 15.5 ± 1.2 | 352.7 ± 6.9 |
| combination | 12.3 ± 0.6 | 13.2 ± 1.4 | 352.0 ± 6.0 |

Mean ± S.E. (n = 8)

At the start of the GTT, blood was collected to measure plasma GLP-1. The plasma glucose and insulin concentrations were measured before and after loading with the glucose solution. For the preparation of the glucose solution, glucose was diluted in saline at a concentration of 0.4 g/mL.

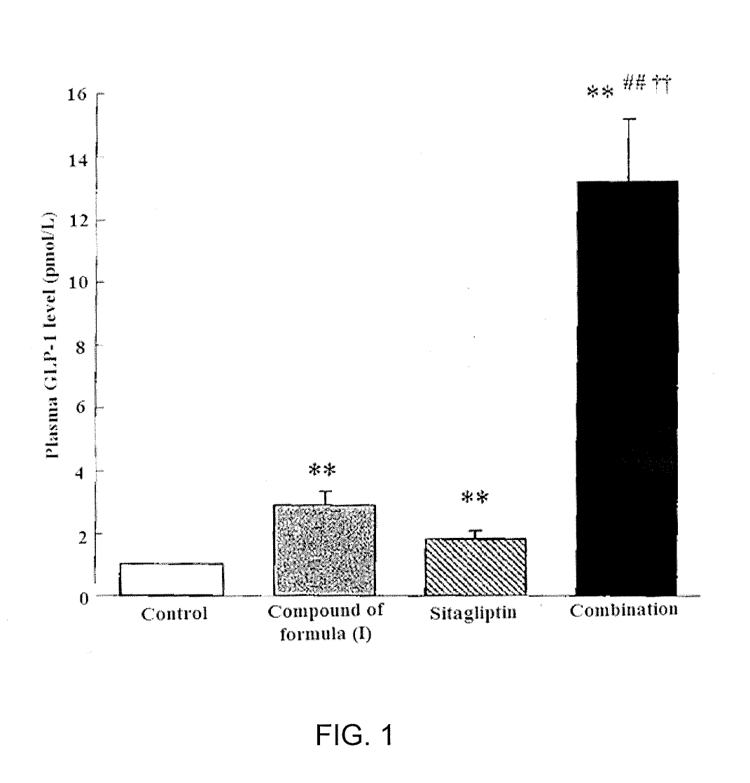

Plasma GLP-1 levels prior to glucose loading in the compound of formula (I) and the sitagliptin groups were significantly higher than those in the control group. Plasma GLP-1 levels in the combination group was markedly increased as compared with those in the compound of formula (I) and the sitagliptin groups (Table 4, FIG. 1).

TABLE 4
Effect on Plasma GLP-1 Level at the Start of the GTT

| Group | Plasma GLP-1 Level (pmol/L) |
|---|---|
| control | 1.024 ± 0.021 |
| compound of formula (I) | 2.883 ± 0.420** |
| sitagliptin | 1.800 ± 0.270** |
| combination | 13.210 ± 1.988**††^{##} |

Mean ± S.E. (n = 8)
**p < 0.01 versus control;
††p < 0.01 versus compound of formula (I);
^{##}p < 0.01 versus sitagliptin: Steel-Dwass test

For the GTT, the rats were fasted overnight after six hours feeding. On the following day, 600-700 μL of blood were collected from the tail vein into tubes containing a DPP-IV inhibitor, aprotinin, and EDTA. Thereafter, a glucose solution at a dose of 1 g/2.5 mL/kg was loaded intraperitoneally. At 10, 30, 60, and 120 minutes after loading the glucose solution, 200-300 μL of blood were collected from the tail vein into tubes containing heparin. The blood was centrifuged at 10,000×g and 4° C. for five minutes to obtain the plasma.

After 200 μL of plasma sampled before the glucose loading were added with 467 of ethanol, the solution was thoroughly mixed. The mixture was separated by centrifugation at 4° C. and 10,000×g for five minutes. The supernatant was evaporated to dryness using a centrifuge evaporator (EC57-CS, Sakuma Seisakusho, Ltd.). The residue was dissolved in 100 μL of assay buffer from a GLP-1 (Active) ELISA kit (Millipore Corporation). The GLP-1 concentration was measured by the ELISA method using a GLP-1 (Active) ELISA kit.

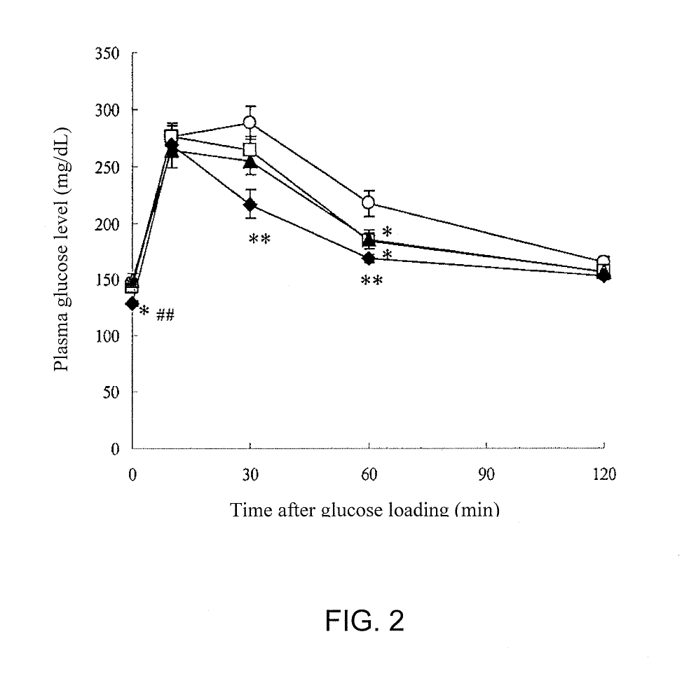

In the GTT, plasma glucose levels in the compound of formula (I) and the sitagliptin groups were lower and significantly decreased at 60 minutes after glucose loading as compared with the control group. In the combination group, plasma glucose levels before glucose loading were lower than those in the other three groups. Plasma glucose levels after glucose loading were lower than those in the compound of formula (I) and the sitagliptin groups. In the compound of formula (I) and sitagliptin groups the area under the curve (AUC) for the glucose levels during the GTT was lower than that for the control group, although there were no statistically significant differences. The AUC in the combination group was lower than those in the other three groups and was significantly decreased as compared with the control group (Table 5, FIG. 2).

TABLE 5
Effect on Plasma Glucose Levels in the GTT
Plasma Glucose Level (mg/dL)

| | | | | | | AUC of Plasma |
|---|---|---|---|---|---|---|
| | Time After Glucose Loading (min) | | | | | Glucose Level |
| Group | 0 | 10 | 30 | 60 | 120 | (mg · min/dL) |
| control | 148 ± 4 | 276 ± 12 | 288 ± 14 | 217 ± 11 | 165 ± 5 | 26796 ± 1082 |
| compound of | 143 ± 3 | 277 ± 9 | 264 ± 12 | 184 ± 7* | 157 ± 3 | 24453 ± 634 |
| formula (I) | | | | | | |
| sitagliptin | 149 ± 6 | 264 ± 16 | 255 ± 12 | 185 ± 8* | 157 ± 5 | 24095 ± 981 |
| combination | 129 ± 2*^{##} | 269 ± 6 | 217 ± 12** | 168 ± 4** | 153 ± 4 | 22251 ± 567** |

Tukey Test -
*p < 0.05;
**p < 0.01 versus control;
^{##}p < 0.01 versus sitagliptin

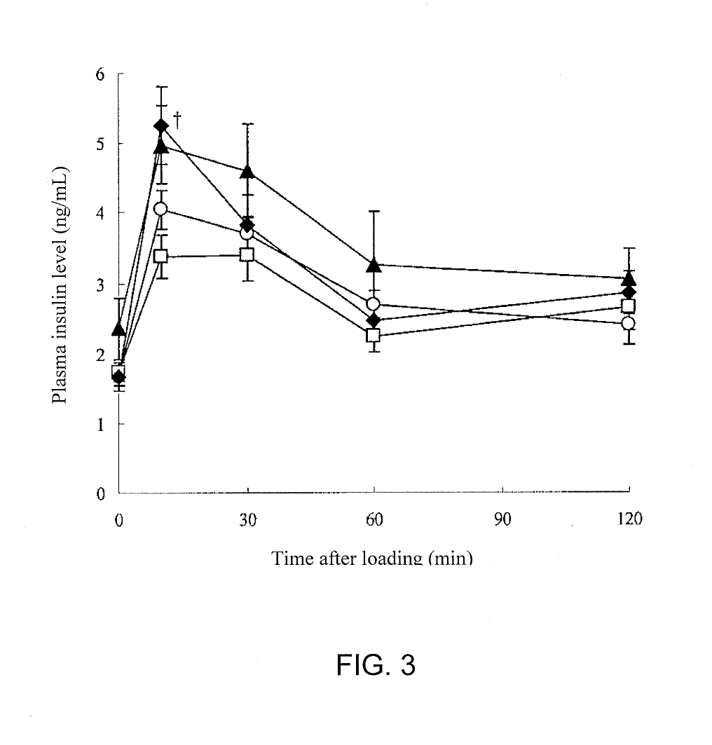

The plasma insulin levels during the GTT in the compound of formula (I) group were lower, while those in the sitagliptin group were higher as compared with the control group. In the combination group, the plasma insulin levels at ten minutes after glucose loading were higher than those in the control and the compound of formula (I) groups, and significantly higher as compared with those in the compound of formula (I) group (Table 6, FIG. 3).

TABLE 6
Effect on Plasma Insulin Levels in the GTT
Plasma Insulin Level (ng/mL)

| | | | | | | AUC of Plasma |
|---|---|---|---|---|---|---|
| | Time After Glucose Loading (min) | | | | | Insulin Level |
| Group | 0 | 10 | 30 | 60 | 120 | (ng · min/mL) |
| control | 1.681 ± 0.139 | 4.038 ± 0.275 | 3.698 ± 0.258 | 2.681 ± 0.202 | 2.400 ± 0.290 | 354.048 ± 22.032 |
| compound of | 1.724 ± 0.114 | 3.378 ± 0.305 | 3.392 ± 0.360 | 2.232 ± 0.222 | 2.635 ± 0.312 | 323.571 ± 30.631 |
| formula (I) | | | | | | |
| sitagliptin | 2.350 ± 0.439 | 4.968 ± 0.562 | 4.595 ± 0.675 | 3.253 ± 0.749 | 3.046 ± 0.435 | 438.898 ± 72.779 |
| combination | 1.666 ± 0.197 | 5.249 ± 0.557^{†} | 3.834 ± 0.426 | 2.456 ± 0.253 | 2.852 ± 0.309 | 379.004 ± 30.898 |

Tukey Test -
^{†}p < 0.05 versus compound of formula (I)

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating or preventing diabetes in a subject comprising administering a therapeutically effective amount of (a) a compound of formula (I) 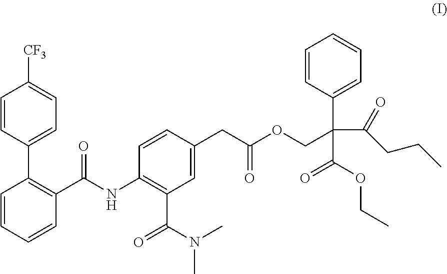 [SMILES: CCCC(=O)C(COC(=O)Cc1ccc(NC(=O)c2ccccc2-c2ccc(C(F)(F)F)cc2)c(C(=O)N(C)C)c1)(C(=O)OCC)c1ccccc1]
or a pharmaceutically acceptable salt thereof and (b) a dipeptidyl peptidase-IV (DPP-IV) inhibitor or a pharmaceutically acceptable salt thereof, to a subject that has or is at risk of having diabetes, whereupon diabetes is treated or prevented in the subject.

2. The method of claim 1, wherein the therapeutically effective amount of the compound of formula (I) is about 1 mg to about 20 mg per day.

3. The method of claim 1, wherein the compound of formula (I) is administered twice per day.

4. The method of claim 1, wherein the method comprises administering twice per day a therapeutically effective amount of the compound of formula (I), or a pharmaceutically acceptable salt thereof, to the subject, and the therapeutically effective amount is increased once during the course of the administration of the compound of formula (I), or a pharmaceutically acceptable salt thereof.

5. The method of claim 4, wherein the method comprises
(a) administering twice per day a first therapeutically effective amount of the compound of formula (I), or a pharmaceutically acceptable salt thereof, to the subject for a first time period, and
(b) administering twice per day a second therapeutically effective amount of the compound of formula (I), or a pharmaceutically acceptable salt thereof, to the subject for a second time period after the first time period,
wherein the second therapeutically effective amount is greater than the first therapeutically effective amount.

6. The method of claim 5, wherein the first time period is about 1 to 5 weeks.

7. The method of claim 5, wherein the first time period is about 2 to 4 weeks.

8. The method of claim 5, wherein the first time period is about 2 weeks.

9. The method of claim 5, wherein
the second therapeutically effective amount of the compound of formula (I) is at least 25% greater than the first therapeutically effective amount of the compound of formula (I).

10.-12. (canceled)

13. The method of claim 1, wherein the method comprises administering twice per day a therapeutically effective amount of the compound of formula (I), or a pharmaceutically acceptable salt thereof, to the subject, and the therapeutically effective amount is increased twice during the course of the administration of the compound of formula (I), or a pharmaceutically acceptable salt thereof.

14. The method of claim 13, wherein the method comprises
(a) administering twice per day a first therapeutically effective amount of the compound of formula (I), or a pharmaceutically acceptable salt thereof, to the subject for a first time period,
(b) administering twice per day a second therapeutically effective amount of the compound of formula (I), or a pharmaceutically acceptable salt thereof, to the subject for a second time period after the first time period, and
(c) administering twice per day a third therapeutically effective amount of the compound of formula (I), or a pharmaceutically acceptable salt thereof, to the subject for a third time period after the second time period,
wherein
the second therapeutically effective amount is greater than the first therapeutically effective amount, and
the third therapeutically effective amount is greater than the second therapeutically effective amount.

15. The method of claim 14, wherein the first and second time periods are each independently about 1 to 5 weeks.

16. (canceled)

17. The method of claim 14, wherein the first and second time periods are about 2 weeks.

18. The method of claim 14, wherein the second therapeutically effective amount of the compound of formula (I) is at least 25% greater than the first therapeutically effective amount of the compound of formula (I).

19.-21. (canceled)

22. The method of claim 14, wherein the third therapeutically effective amount of the compound of formula (I) is at least 25% greater than the second therapeutically effective amount of the compound of formula (I).

23.-25. (canceled)

26. The method of claim 1, wherein the diabetes is type 2 diabetes.

27. The method of claim 1, wherein the compound of formula (I) and the DPP-IV inhibitor are administered to the subject when the subject is in the fed state.

28. The method of claim 27, wherein the compound of formula (I) and the DPP-IV inhibitor are administered to the subject with food.

29. The method of claim 27, wherein the compound of formula (I) and the DPP-IV inhibitor are administered to the subject 1-180 minutes after the subject has begun to consume food.

30.-36. (canceled)

37. The method of claim 1, wherein the DPP-IV inhibitor or a pharmaceutically acceptable salt thereof is selected from the group consisting of sitagliptin, vildagliptin, saxagliptin, linagliptin, dutogliptin, gemigliptin, and alogliptin.

38. The method of claim 37, wherein the DPP-IV inhibitor or a pharmaceutically acceptable salt thereof is sitagliptin.

39.-43. (canceled)
